(19) United States
(12) Patent Application Publication
(10) Pub. No.: US 2011/0201588 A1
(43) Pub. Date: Aug. 18, 2011

(54) Post Foaming Gel Composition Comprising An Anti-Irritation Agent

(76) Inventor: James Robert Schwartz, West Chester, OH (US)

(21) Appl. No.: 13/028,865

(22) Filed: Feb. 16, 2011

Related U.S. Application Data
(60) Provisional application No. 61/305,129, filed on Feb. 16, 2010.

(51) Int. Cl. A61K 8/58; A61Q 9/02; A61K 8/81; A61K 8/87; A61K 8/73; A61Q 19/00

(52) U.S. Cl. 514/188

(57) ABSTRACT

A personal care composition which is preferably a post-foaming gel, said composition comprising an anti-irritation agent; a water dispersible surface active agent, of a carrier comprising water; and optionally a lubricant.

### CROSS REFERENCE TO RELATED APPLICATION

This application claims the benefit of U.S. Provisional Application No. 61/305,129, filed Feb. 16, 2010.

### FIELD OF THE INVENTION

The present invention relates to a personal care composition, preferably one in the form of an aerosol shaving gel or foam that contains an anti-irritation agent. The personal care composition is preferably a shave or hair removal preparation.

### BACKGROUND OF THE INVENTION

One of the more popular forms used today is the post foaming shave gel, developed in the late 70's. Amine-based soaps are combined with volatile hydrocarbons to form a clear, stable emulsion when kept under pressure. Once dispensed and mechanically agitated, these gels transform into thick foams.

Currently, a widely used form of shaving preparation is the type referred to as a post-foaming shave gel. These post-foaming shave gels are now well-known. See, e. g., U.S. Pat. Nos. 5,326,556 and 5,500,211. Various attempts have been made to increase the lubricity of shaving compositions. Even as many of these systems provide lubrication and/or a smooth shave, skin irritation remains a problem. A number of approaches to reduce irritation have been attempted, but have not resulted in satisfactory results. Moreover, as skin irritation indicates damage to the skin, the skin is left more susceptible to microbial infection. Thus, there is a need to treat the irritation of the skin as well as treat the skin for antimicrobials.

### SUMMARY OF THE INVENTION

One aspect of this invention relates to a personal care composition which is in the form of an aerosol product, preferably a post foaming gel or a shaving foam, said composition comprising: from about 0.01% to 5% of an anti-irritation agent; from about 2% to about 25% of a water dispersible surface active agent; from about 60% to about 93% of a carrier comprising water; and a lubricant.

### DETAILED DESCRIPTION OF THE INVENTION

The term “fatty”, as used herein, means a hydrocarbon chain having 12-22 carbon atoms (C12-22), preferably 14-18 carbon atoms (C14-18). The chain may be straight or branched and may be saturated or unsaturated (typically one or two double bonds in the chain). The term “water dispersible”, as used herein, means that a substance is either substantially dispersible or soluble in water.

The personal care composition of the present invention is suitable for use as a hair removal preparation, such as a post-foaming shave gel composition. In one embodiment the composition comprises from about 0.005% to about 3% of a cationic polysaccharide, wherein said cationic polysaccharide is hydrophobically modified; about 2% to about 25%, preferably about 5% to about 20%, of a water dispersible surface active agent, from about 60% to about 93%, or from about 70% to about 85% of a carrier, such as water; and a lubricant. The lubricant can comprise from about 0.01% to about 1%, of a lubricious water soluble polymer, from about 0.01% to about 5%, preferably about 0.1% to about 2%, of water insoluble particles, and about 0.0005% to about 3%, preferably about 0.001% to about 0.5%, of a hydrogel-forming polymer, by weight of the composition respectively. Preferably, the composition is in the form of a post-foaming shave gel and will additionally include from about 1% to about 6%, preferably from about 2% to about 5%, of a volatile post-foaming agent.

Without wishing to be bound by theory, it is believed that the compositions of the present invention surprisingly provide an antimicrobial benefit and reduce irritation. It is believed that zinc pyrithione, when added to these compositions, provides the synergistic benefit of reduced irritation, while providing an antimicrobial benefit. It is this unique combination of benefits provided by these compositions that result in an improved shaving experience.

### 1. Anti-Irritation Agent

In the personal care composition of the present invention, the anti-irritation agent is pyrithione or a polyvalent metal salt of pyrithione. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

The composition of the present invention optionally includes an effective amount of a zinc salt.

Preferred embodiments of the present invention include an effective amount of a zinc salt having an aqueous solubility within the composition of less than about 25%, by weight, at 25° C., more preferably less than about 20%, more preferably less than about 15%.

Preferred embodiments of the present invention include from 0.001% to 10% of a zinc salt, more preferably from 0.01% to 5%, more preferably still from 0.1% to 3%.

In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

### 2. Water Dispersible Surface Active Agent

The water dispersible surface active agent is preferably one that is capable of forming a lather and may comprise a soap, an interrupted soap, a detergent, an anionic surfactant, a non-ionic surfactant or a mixture of one or more of these. One group of suitable water dispersible surface active agent are lathering surfactants, such as those selected from the group consisting of anionic surfactants, nonionic surfactants, amphoteric surfactants, zwiterrionic surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants

Concentrations of these surfactants are from about 1% to about 20%, alternatively from about 5% to about 25%, and alternatively from about 2% to about 30% by weight of the composition.

Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth-20, Steareth-21, Ceteth-20, Laureth-4 and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, lauryl polyglucoside, sucrose laurate, and polyglycerol 8-oleate. Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Suitable amphoteric surfactants include, for example, the betaines and sultaines such as cocoamidopropyl betaine, coco dimethyl carboxymethyl betaine, coco sultaine and the like. Non-limiting examples of suitable zwitterionic or amphoteric surfactants are described in U.S. Pat. Nos. 5,104,646 and 5,106,609.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. Suitable anionic lathering surfactants include, for example, the sodium, potassium, ammonium and substituted ammonium salts (such as the mono-, di- and triethanolamine salts) of C8-C22, preferably C12-C18, alkyl sulfates (e.g., sodium lauryl sulfate, ammonium lauryl sulfate), alkyl sulfonates (e.g., ammonium lauryl sulfonate), alkylbenzene sulfonates (e g ammonium xylene sulfonate), acyl isethionates (e.g. sodium cocoyl isethionate), acyl lactylates (e.g. sodium cocoyl lactylate) and alkyl ether sulfates (e.g., ammonium laureth sulfate).

The water dispersible surface active agent can also include soaps, such as the sodium, potassium and lower alkanolamine (preferably triethanolamine) salts of C12-22, preferably C14-18, fatty acids. Typical fatty acids include lauric, myristic, palmitic and stearic acid and mixtures thereof. The preferred fatty acids are palmitic and stearic. The interrupted soaps include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in preneutralized form (i.e., as the sodium, potassium or alkanolamine salt) or in the free acid form followed by subsequent neutralization with sodium hydroxide, potassium hydroxide and/or lower alkanolamine (preferably triethanolamine). In any event, the final composition must contain sufficient base to neutralize or partially neutralize the soap component and adjust the pH to the desired level (typically between 5 and 10, more typically between 6 and 9). It is most preferred that the composition of the present invention includes a soap (e.g., triethanolamine palmitate/stearate) or an interrupted soap (e.g., triethanolamine stearoyl/myristoyl sarcosinate), or a mixture thereof.

In one embodiment, the composition is free or essentially free of soap. As used herein, “essentially free” of a component means that no amount of that component is deliberately incorporated into the composition. In one embodiment the composition is a self-foaming soap free shave gel as described in U.S. Pat. No. 5,500,211.

### 3. Lubricant

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Particularly suitable polyethylene oxides include, for example, PEG-14M (MW≅600,000) PEG-23M (MW≅1 million), PEG-45M (MW≅2 million) and PEG-90M (MW≅4 million). The lubricious water soluble polymer will generally be included in the post foaming gel composition in an amount of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles. Any of the forgoing particles may also include a surface treatment to make the particles more readily dispersible or improve their cosmetic aesthetics. Preferred are polytetrafluoroethylene particles (e.g., PTFE particles available from MicroPowders, Inc. under the tradename Microslip). Preferably the water insoluble particles will have an average particle size of about 1 μm to about 100 μm, more preferably about 2 μm to about 50 μm, and most preferably about 5 μm to about 15 μm. The particles may be of any desired shape including spherical bead, elongated fiber or irregular shape, with spherical bead being the preferred shape. Generally the water insoluble particles will be included in the post foaming gel composition in an amount of about 0.01% to about 5%, preferably about 0.1% to about 2%, by weight.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. Generally, the hydrogel-forming polymer will be included in the post foaming gel composition in an amount of about 0.0005% to about 3%, preferably about 0.001% to about 0.5%, more preferably about 0.002% to about 0.1%, by weight.

A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin. A preferred source of glyceryl acrylate/acrylic acid copolymer is available from ISP Technologies, Inc. (United Guardian Inc.) under the tradename Lubrajel®, particular the form known as Lubrajel® oil which contains about 1.0%-1.3% glyceryl acrylate/acrylic acid copolymer in aqueous glycerin (⁻40% glycerin). Lubrajel® oil also includes about 0.6% PVM/MA copolymer (also known as methoxyethylene/maleic anhydride copolymer), which may further contribute to the lubricity of this source. Most preferably, the post foaming gel composition will include about 0.25% to about 4% Lubrajel® oil in order to provide a preferred level of about 0.002% to about 0.05% of the glyceryl acrylate/acrylic acid copolymer. This amount of Lubrajel® oil will also provide about 0.001% to about 0.03% of PVM/MA copolymer.

### 4. Post Foaming Agent

The post-foaming agent, when included in the post foaming gel composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig. The post-foaming agent will be present in an amount to provide the post foaming gel composition with a sufficiently rapid turnover—that is, transition from gel to foam when contacted with the skin—typically, in about 2 to about 30 seconds, preferably in about 5 to about 15 seconds.

### 5. Carrier

The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the post foaming gel composition comprises from about 50% to about 99.99%, preferably from about 60% to about 93%, more preferably from about 70% to about 90%, and even more preferably from about 80% to about 85% of the carrier by weight of the composition. In one embodiment, the carrier comprises water.

### 6. Other Adjunct Ingredients

Although not necessary to forming a useful shave gel composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, etc. It is particularly preferred to include glycerin in the shave gel composition of the present invention, preferably in an amount of about 0.1% to about 3%, more preferably about 0.3% to about 1%, by weight. Glycerin improves the emolliency of the composition.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); Crosslinked Polyacrylate Polymers (including both cationic and nonionic polymers, such as described in U.S. Pat. Nos. 5,100,660; 4,849,484; 4,835,206; 4,628,078; 4,599,379, and EP 228,868); Polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); Polyacrylamide Polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); Polysaccharides (nonlimiting examples of polysaccharide gelling agents include those selected from the group consisting of cellulose, carboxymethyl hydroxyethylcellulose (sold under the trademarks “Natrosol”), cellulose acetate propionate carboxylate, hydroxyethylcellulose, hydroxyethyl ethylcellulose, hydroxypropylcellulose (sold under the trademarks “Klucel”), hydroxypropyl methylcellulose, methyl hydroxyethylcellulose, microcrystalline cellulose, sodium cellulose sulfate, and mixtures thereof); Gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. No. 6,967,027 to Heux et al.; U.S. Pat. No. 5,207,826 to Westland et al.; U.S. Pat. No. 4,487,634 to Turbak et al.; U.S. Pat. No. 4,373,702 to Turbak et al. and U.S. Pat. No. 4,863,565 to Johnson et al., U.S. Patent Publ. No. 2007/0027108 to Yang et al.)

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof. Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and U.S. 2005/0019356 (i.e. desquamation actives, anti-acne actives, chelators, flavonoids, and antimicrobial and antifungal actives). Examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed. Other useful optional ingredients include: Anti-Wrinkle Actives and/or Anti-Atrophy Actives; Anti-Oxidants and/or Racial Scavengers; Anti-Inflammatory Agents; Anti-Cellulite Agents; Tanning Actives; Skin Lightening Agents; Sunscreen Actives; Water Soluble Vitamins; particulates; and combinations thereof.

The composition can also include other commonly included ingredients which are used in commercially available post foaming shave gels such as those described in US Patent Publ. Nos. 2006/0257349, 2006/0257350 and 2005/0175575.

### 7. Product Forms and Uses

The personal care compositions of the present invention can be used for as a hair removal preparation such as a post foaming shave gel. The present composition may be formulated as an aerosol foam, a post-foaming gel (which is the preferred form) or a non-aerosol gel or lather. It may be packaged in any suitable dispenser normally used for dispensing personal care compositions (such as shaving compositions). These include collapsible tubes, pump or squeeze containers, and aerosol-type dispensers, particularly those with a barrier to separate the post foaming gel composition from the propellant required for expulsion.

The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) manually activated air pump spray devices in which a pump system is integrated into the container to allow the user to pressurize the container with air in order to expel the product (e.g., the “AIRSPRAY” system available from Airspray International); (3) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (4) bag-in-can (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

### 8. In Shave Lubrication Test

It has been found that the personal care composition of the present invention provides for an in shave lubrication benefit as shown by reduced friction as measured by the In Shave Lubrication “ISL” Test defined herein. Reducing friction is important during the shave because a high friction skin surface results in bulging of the skin When the skin bulges, the blade is more likely to engage the skin, increasing the chance for skin irritation. Therefore, by reducing friction the product helps protect the skin. In addition, lower friction results in less drag on the skin, which can also be a potential source of irritation. This method enables measurement of the coefficient of friction (CoF) of a shave preparation.

In Shave Lubrication Test Method: An apparatus designed to simulate lubrication during the shaving process is connected to an instrument capable of measuring frictional forces (for example, an Instron-type instrument) and containing a load cell of about 1 kg to about 100 kg. The rinsing apparatus comprises: 1) an air-activated clamping device capable of opening and closing to deliver pressures of about 10 psi to about 70 psi to simulate the pressure exerted by hands on hair during rinsing 2) keratinous tissue models as described herein affixed to two opposing sides of the clamping device and 3) one or more spray nozzles capable of delivering water flow rates of from about 50 ml/min to about 1000 mL/min., for simulating shower conditions.

Procedure: Attach the rinsing apparatus to the base of a Stable Micro Systems TA XT Pus™ Texture Analyzer (TA) equipped with a 30 kg load cell, centering or aligning the clamps perpendicular to the load cell. Adjust water flow rate to approximately 200 ml/min and the water temperature to 103° F.+/−2° F. Set the air pressure for the TA clamps to approximately 30 psi. Set the instrument measurement settings as follows: TA settings, tension compression, test speed—10.0 mm/sec for 130 mm long pull. Set the macro for a total of 10 strokes. Run the first five strokes without the water on, then manually turn on the water for 2 min 15 sec for the second five strokes. During the test, data (g of force) will only be collected during the upward pull of the treated KTM, not on the return. Cover the pads on both the front and back side of the piston with a polyurethane skin pad (see JP2006233367 for details).

Wet a 2 inch by 9 inch piece of nonwoven KTM under hot (−103° F.+/−2° F.) tap water for 30 seconds. Place 2 g+/−0.1 g of aerosol shave gel or 1 g+/−0.1 g of aerosol shave foam onto the nonwoven KTM and gently lather and spread by hand for 30 seconds. Rub excess foam on the back of the nonwoven KTM. Load the nonwoven KTM into the TA and start the test macro. At the end of the fifth stroke, turn on the rinse water. Initiate a test sequence which 1) instructs the instrument to raise the load cell to which the KTM is attached, at a rate of about 10 mm/sec 2) opens the clamps, and 3) instructs the instrument to lower the load cell. Repeat this sequence until a predetermined number of sequences may be executed, for example, 10. Between each sample, wipe the polyurethane skin pads with a piece of nonwoven KTM and an alcohol wipe to remove any possible build-up from the previous test. By calculating the total friction in grams of force (or other suitable unit of force) for dry friction and rinse friction, products may be ranked relative to each other to assess which products would be expected to have the most pleasant feel.

“KTM” as defined herein means a “Keratinous tissue mimic” which refers to one or more artificial substrates which may have one or more physical properties representative of keratinous tissue. The KTM used for the purposes of this application is TENCEL from Lenzing, Inc. Additional details on other KTMs is disclosed in Section I of U.S. Ser. No. 61/239,908 to Battaglia et al, filed Sep. 4, 2009.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited herein are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a”, “an”, and “the” mean “one or more”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol shave composition comprising:
a. from about 0.01% to 5% of an anti-irritation agent;
b. from about 2% to about 25% of a water dispersible surface active agent; and
c. from about 60% to about 93% of a carrier comprising water.

2. The aerosol shave composition of claim 1, further comprising from about 1% to about 6% of a volatile post-foaming agent.

3. The aerosol shave composition of claim 1, wherein the aerosol shave composition is a post foaming shave gel or an aerosol foam.

4. The aerosol shave composition of claim 1, wherein said aerosol shave composition comprises from about 0.25% to about 2.5% of an anti-irritation agent.

5. The aerosol shave composition of claim 1, wherein said anti-irritation agent comprises zinc pyrithione.

6. The aerosol shave composition of claim 1, wherein said aerosol shave composition further comprises from 0.001% to 10% of a zinc salt.

7. The aerosol shave composition of claim 6, wherein said zinc salt comprises zinc oxide.

8. The aerosol shave composition of claim 1, further comprising a lubricant.

9. The aerosol shave composition of claim 8, wherein said lubricant comprises at least one of: a lubricious water soluble polymer, a water insoluble particle, a hydrogel-forming polymer, and a mixture thereof.

10. The aerosol shave composition of claim 9, wherein said lubricant comprises at least one of: from about 0.01% to about 1%, of said lubricious water soluble polymer, from about 0.01% to about 5% of said water insoluble particle, from about 0.0005% to about 3%, of said hydrogel-forming polymer, and a mixture thereof, by weight of the aerosol shave composition.

11. The aerosol shave composition of claim 8, wherein said lubricious water soluble polymers comprises at least one of a polyethylene oxide, a polyvinylpyrrolidone, a polyacrylamide, and a mixture thereof.

12. The aerosol shave composition of claim 8, wherein said water insoluble particles comprises at least one of an inorganic particles, an organic polymer particles, and a mixture thereof.

13. The aerosol shave composition of claim 8, wherein said hydrogel-forming polymers comprises at least one of the following: a polyacrylic acid or polymethacrylic acid partially esterified with a polyhydric alcohol; a hydrophilic polyurethanes; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

14. The aerosol shave composition of claim 13, wherein said hydrogel-forming polymer comprises polyacrylic acid partially esterified with glycerin.

15. The aerosol shave composition of claim 1, wherein said water dispersable surface active agent comprises a lathering surfactant other than an non-ionic surfactant.

16. The aerosol shave composition of claim 1, wherein said water dispersable surface active agent is free or essentially free of a soap.

17. The aerosol shave composition of claim 1, further comprising a humectant.
